(19) United States
(12) Patent Application Publication
(10) Pub. No.: US 2022/0280226 A1
(43) Pub. Date: Sep. 8, 2022

(54) SEGMENTED SURGICAL FORCEPS

(71) Applicant: GYRUS ACMI, INC. D/B/A OLYMPUS SURGICAL TECHNOLOGIES AMERICA, Westborough, MA (US)

(72) Inventors: Thomas J. Holman, Princeton, MN (US); Nikhil M. Murdeshwar, Maple Grove, MN (US)

(21) Appl. No.: 17/652,959

(22) Filed: Mar. 1, 2022

Related U.S. Application Data
(60) Provisional application No. 63/155,561, filed on Mar. 2, 2021.

(51) Int. Cl. A61B 18/14

(52) U.S. Cl. CPC A61B 18/1445; A61B 2018/1452; A61B 2018/00589

(57) ABSTRACT

A forceps can include a first jaw and a second jaw. The first jaw can include a first jaw frame and a first movable jaw segment coupled with the first jaw frame. The second jaw can be coupled with the first jaw and the first movable jaw segment can be in communication with a first electromagnetic actuation system. The first movable jaw segment is configured to be electromagnetically actuatable relative to the first jaw frame.

### CLAIM OF PRIORITY

This patent application claims the benefit of priority, under 35 U.S.C. Section 119(e), to Thomas J. Holman U.S. Patent Application Ser. No. 63/155,561, entitled “SEGMENTED SURGICAL FORCEPS,” filed on Mar. 2, 2021, which is hereby incorporated by reference herein in its entirety.

### BACKGROUND

Medical devices for diagnosis and treatment, such as forceps, are often used for medical procedures such as laparoscopic and open surgeries. Forceps can be used to manipulate, engage, grasp, or otherwise affect an anatomical feature, such as a vessel or other tissue of a patient during the procedure. Forceps often include an end effector that is manipulatable from a handle of the forceps. For example, jaws located at a distal end of a forceps can be actuated via elements of the handle between open and closed positions to thereby engage the vessel or other tissue. Forceps can include an extendable and retractable blade that can be extended distally between a pair of jaws to lacerate the tissue. The handle can also be capable of supplying an input energy, such as electromagnetic energy or ultrasound, to the end effector for sealing of the vessel or tissue during the procedure.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a side view of a forceps showing jaws in an open position.

FIG. 2A illustrates an isometric view of a portion of forceps in a closed position.

FIG. 2B illustrates an isometric view of a portion of forceps in a partially open position.

FIG. 2C illustrates an isometric view of a portion of forceps in an open position.

FIG. 3A illustrates a side view of a portion of forceps in a first configuration.

FIG. 3B illustrates a side view of a portion of forceps in a second configuration.

FIG. 4 illustrates a side view of a portion of forceps.

FIG. 5 illustrates a side view of a portion of forceps.

FIG. 6 illustrates a side view of a portion of forceps.

FIG. 7A illustrates a side view of a portion of forceps.

FIG. 7B illustrates a top view of a portion of forceps.

FIG. 8 illustrates a side view of a portion of forceps.

FIG. 9A illustrates a side view of a portion of forceps in a first configuration.

FIG. 9B illustrates a side view of a portion of forceps in a second configuration.

FIG. 10 illustrates a schematic of a control system.

FIG. 11 illustrates a block diagram of a machine upon which one or more embodiments may be implemented.

### DETAILED DESCRIPTION

Electrosurgical forceps are often used for grasping and cutting tissue. When the tissue, such as a vessel is grasped or grabbed by opposing jaws of a forceps, the vessel can be compressed unevenly due to the mechanics of the jaws—the jaws pivot at the (proximal) end opposite the open (distal) end. That is, a proximal portion of the jaw will engage the tissue or vessel before a distal portion of the jaw will engage the vessel or tissue, which can result in uneven forces applied to the tissue or vessel. Further, pressure applied to the tissue or vessel can be applied by the jaws unevenly (more pressure applied by the proximal jaw portions). Such uneven pressure and engagement can result in the tissue being squeezed out of the jaw or being misshapen by the jaws, which can impact cutting performance. For example, a vessel can balloon outward from the jaw, which can cause an incomplete cutting and sealing of the vessel.

The present disclosure can help to address these issues by including a jaw with secondary moving components. For example, segments of the jaw can be movable with respect to frames of the jaw to help apply homogeneous pressure to tissues and vessels. Electrodes can be optionally movable relative to the frames or the jaws for additional engagement with the tissues or vessels to help apply homogenous pressure to the tissues and vessels. Such components can help to improve cutting and sealing operations using surgical forceps during procedures.

The above discussion is intended to provide an overview of subject matter of the present patent application. It is not intended to provide an exclusive or exhaustive explanation of the invention. The description below is included to provide further information about the present patent application.

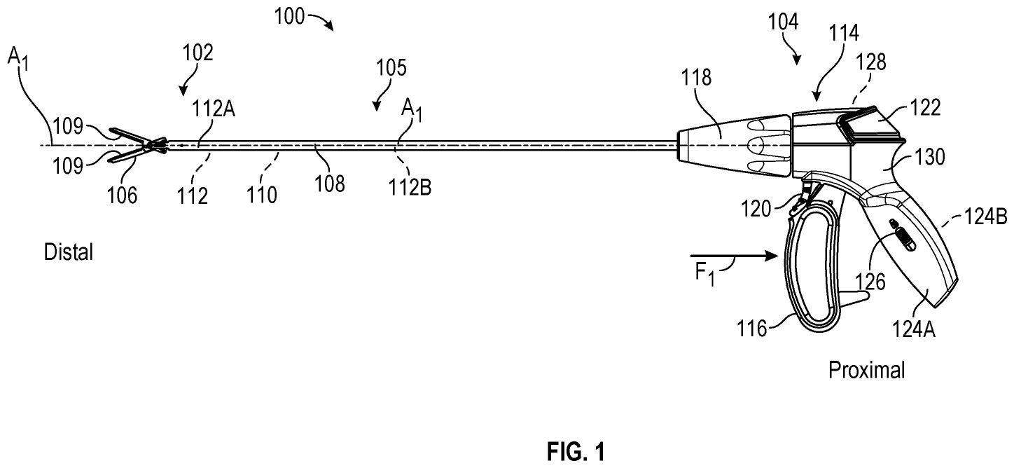

FIG. 1 illustrates a side view of a forceps 100 showing jaws in an open position. The forceps 100 can include an end effector 102, a handpiece 104, and an intermediate portion 105. The end effector 102 can include jaws 106 (including electrodes 109), an outer shaft 108, an inner shaft 110, and a blade assembly 112. The handpiece 104 can include a housing 114, a lever 116, a rotational actuator 118, a trigger 120, an activation button 122, a fixed handle 124a and 124b, and a handle locking mechanism 126. The housing 114 can include a first housing portion 128, and a second housing portion 130. FIG. 1 also shows orientation indicators Proximal and Distal and a longitudinal axis A1.

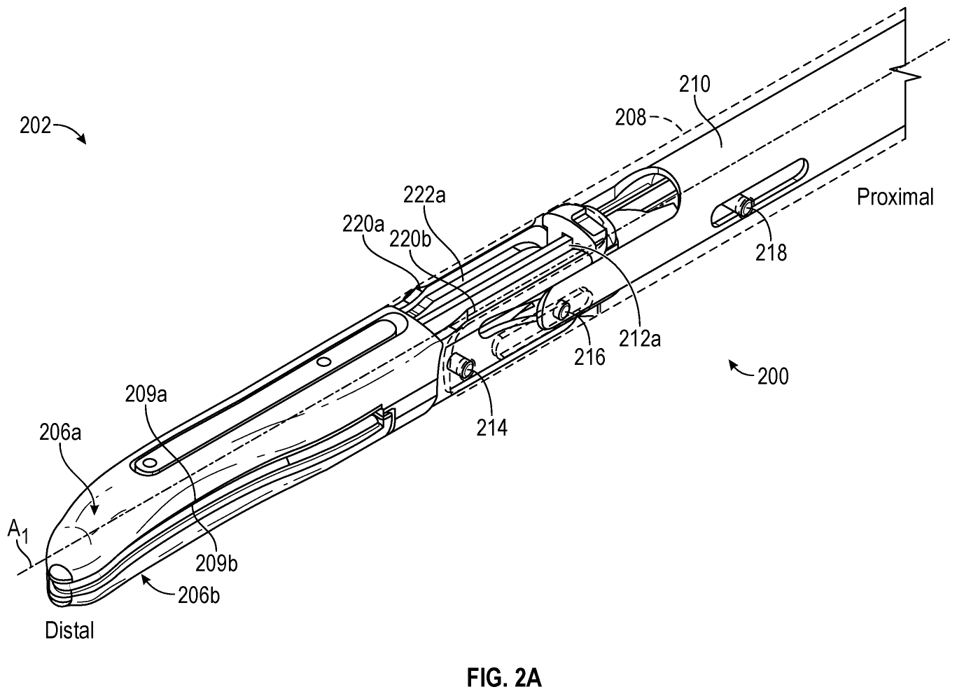

Generally, the handpiece 104 can be located at a proximal end of the forceps 100 and the end effector 102 can be located at the distal end of the forceps 100. The intermediate portion 105 can extend between the handpiece 104 and the end effector 102 to operably couple the handpiece 104 to the end effector 102. Various movements of the end effector 102 can be controlled by one or more actuation systems of the handpiece 104. For example, the end effector 102 can be rotated along the longitudinal axis A1 (FIG. 2A) of the forceps 100. Also, the handpiece can operate the jaws 106, such as by moving the jaws 106 between open and closed positions. The handpiece 104 can also be used to operate the blade assembly 112 for cutting tissue and can operate the electrode 109 for applying electromagnetic energy to tissue. The end effector 102, or a portion of the end effector 102, can be one or more of: opened, closed, rotated, extended, retracted, and electromagnetically energized.

The housing 114 can be a frame that provides structural support between components of the forceps 100. The housing 114 is shown as housing at least a portion of the actuation systems associated with the handpiece 104 for actuating the end effector 102. However, some or all of the actuation components need not be housed within the housing 114. The housing 114 can provide a rigid structure for attachment of components, but the housing 114 does not necessarily house the components completely, or can house a portion of one or more of the components.

The drive shaft 110 can extend through the housing 114 and out of a distal end of the housing 114, or distally beyond housing 114. The jaws 106 can be connected to a distal end of the drive shaft 110. The outer shaft 108 can be a hollow tube positioned around the drive shaft 110. A distal end of the outer shaft 108 can be located adjacent the jaws 106. The distal ends of the drive shaft 110 and the outer shaft 108 can be rotationally locked to the jaws 106. The rotational actuator 118 can be positioned around the distal end of the housing 114. The outer shaft 108 can extend distally beyond the rotational actuator 118. The blade shaft 112b can extend through the drive shaft 110 and the outer shaft 108. A distal end of the blade shaft 112b can be located near the jaws 106. A proximal end of the blade shaft 112b can be within housing 114.

The handpiece 104 can enable a user to extend and retract a blade 112a of the blade assembly 112, which can be attached to a distal end of a blade shaft 112b of the blade assembly 112. In some examples, the blade 112a can extend an entirety of a length between the handle 104 and the end effector 102. In some examples, the handpiece 104 can include features that inhibit the blade assembly 112 from being extended until the jaws 106 are at least partially closed, or fully closed. The blade 112a can be extended by displacing the trigger 120 proximally and the blade 112a can be retracted by allowing the trigger 120 to return distally to a default position.

A proximal portion of the trigger 120 can be connected to the blade shaft 112b within the housing 114 and a distal portion of the trigger 120 can extend outside of the housing 114 adjacent to, and in some examples nested with, the lever 116 in the default or unactuated positions. The activation button 122 can be coupled to the housing 114 and can include or be connected to electronic circuitry within the housing 114. Such circuitry can send or transmit electromagnetic energy through forceps 100 to the jaws 106. In some examples, the electronic circuitry may reside outside the housing 114 but can be operably coupled to the housing 114 and the end effector 102.

In operation of the forceps 100, a user can displace the lever 116 proximally by applying a Force F1 to the lever 116 to actuate the drive shaft 110 to drive the jaws 106 from the open position (FIG. 2C) to the closed position (FIG. 2A), which can allow the user to clamp down on and compress a tissue. The handpiece 104 can also allow a user to rotate the rotational actuator 118 to cause the end effector 102 to rotate, such as by rotating both the drive shaft 26 and the outer shaft 28 together.

In some examples, with the tissue compressed, a user can depress the activation button 122 to cause an electromagnetic energy, or in some examples, ultrasound, to be delivered to the end effector 102, such as to the electrode 109 and to the tissue. Application of such energy can be used to seal or otherwise affect the tissue being clamped. In some examples, the electromagnetic energy can cause tissue to be coagulated, sealed, ablated, or can cause controlled necrosis. When desired, the trigger 120 can be moved to translate the blade assembly 112 distally such that the blade 112a can extend between the jaws 106 in order to cut the tissue within the jaws 106. Such a process can be repeated, as desired.

In some examples, the forceps 100, or other medical device, may not include all the features described or may include additional features and functions, and the operations may be performed in any order. The handpiece 104 can be used with a variety of other end effectors to perform other methods.

FIG. 2A illustrates an isometric view of a portion of forceps 200 in a closed position. FIG. 2B illustrates an isometric view of a portion of the forceps 200 in a partially open position. FIG. 2C illustrates an isometric view of a portion of the forceps 200 in an open position. FIGS. 2A-2C are discussed below concurrently.

The forceps 200 can include an end effector 202 that can be connected to a handle (such as the handle 104). The end effector 202 can include jaws 206a and 206b, an outer shaft 208, grip plates 209a and 209b, an inner shaft 210, a blade assembly 212, a pivot pin 214, a drive pin 216, and a guide pin 218. The jaw 206a can include flanges 220a and 220b, and the jaw 206b can include flanges 222a and 222b. The grip plate 209a can include a blade slot 224a and the grip plate 209b can include a blade slot 224b. The blade assembly 212 can include a blade 212a and a shaft 212b. FIGS. 2A-2C also show orientation indicators Proximal and Distal and a longitudinal axis A1.

The components of the forceps 200 can each be comprised of materials such as one or more of metals, plastics, foams, elastomers, ceramics, composites, combinations thereof, or the like. Materials of some components of the forceps 200 are discussed below in further detail. Optionally, the forceps 200 (and those discussed below) can be single-use or disposable forceps. Optionally, the forceps 200 (and those discussed below) can be reusable (e.g., autoclavable or sterilizable) forceps.

The jaws 206a and 206b can be rigid or semi-rigid members configured to engage tissue. The jaws 206a and 206b can be coupled to the outer shaft 208, such as pivotably coupled, via the pivot pin 214. The pivot pin 214 can extend through a portion of the jaws 206a and 206b (such as a bore of each of the jaws 206a and 206b) such that the pivot pin 214 can be received by outer arms of the outer shaft 208. In other examples, the jaws 206a and 206b can be pivotably coupled to the outer shaft 208 via a boss or bosses of the outer shaft 208. In another example, the jaws 206a and 206b can include a boss (or bosses) receivable in bores of the outer shaft 208 to pivotably couple the jaws 206a and 206b to the outer shaft 208. In another example, outer shaft 208 can include a boss (or bosses) receivable in bores of the jaws 206a and 206b to pivotably couple the jaws 206a and 206b to the outer shaft 208.

The flanges 220a and 220b (which can be a set of flanges, that is, two flanges) can be rigid or semi-rigid members located at a proximal portion of the jaw 206a. Similarly, the flanges 222a and 222b can be rigid or semi-rigid members located at a proximal portion of the jaw 206b. In some examples, the flanges 220 can be positioned laterally outward of the inner flanges 222. In other examples, the flanges 220 and 222 can be interlaced.

The grip plates 209a and 209b of the jaws 206a and 206b can each be a rigid or semi-rigid member configured to engage tissue and/or the opposing jaw to grasp tissue, such as during an electrosurgical procedure. One or more of the grip plates 209a and 209b can include one or more of serrations, projections, ridges, or the like configured to increase engagement pressure and friction between the grip plates 209a and 209b and tissue. The flanges 220 of the upper jaw 206a can extend proximally away from the grip plate 209a and 209b, and in some examples, substantially downward when the upper jaw 206a is in the open and partially open positions (as shown in FIGS. 2B and 2C, respectively). Similarly, the flanges 222 of the lower jaw 206b can extend proximally away from the grip plate, and in some examples, substantially upward when the upper jaw 206a is in the open and partially open positions (as shown in FIGS. 2B and 2C, respectively), such that the jaws 206a and 206b and flanges 220 and 222 operate to open and close in a scissoring manner. The jaws 206a and 206b can each include an electrode configured to deliver electricity or electrosurgical energy to tissue (optionally through the grip plates 209a and 209b), and a frame supporting the electrode. The blade slots 224a and 224b of the grip plates 209a and 209b can together be configured to receive a blade between the jaws 206a and 206b, when the jaws are moved out of the open position. In some examples, only one blade slot may be used.

Each of the inner shaft 210 and the outer shaft 208 can be a rigid or semi-rigid and elongate body having a geometric shape of a cylinder, where the shape of the inner shaft 210 matches or is generally similar to the shape of the outer shaft 208. In some examples, the inner shaft 210 and the outer shaft 208 can have other shapes such as an oval prism, a rectangular prism, a hexagonal prism, an octagonal prism, or the like. In some examples, the shape of the inner shaft 210 can be different from the shape of the outer shaft 208.

The inner shaft 210 can extend substantially proximally to distally along the axis A1, which can be a longitudinal axis. In some examples, the axis A1 can be a central axis. Similarly, the outer shaft 208 can extend substantially proximally to distally along the axis A1. In some examples, the axis A1 can be a central axis of one or more of the inner shaft 210 and the outer shaft 208. The inner shaft 210 can include an axial bore extending along the axis A1. The outer shaft 208 can also include an axial bore extending along the axis A1. The inner shaft 210 can have an outer dimension (such as an outer diameter) smaller than an inner diameter of the outer shaft 208 such that the inner shaft 210 can be positioned within the outer shaft 208 and such that the inner shaft 210 can be translatable in the outer shaft 208 along the axis A1. The inner shaft 210 can also be referred to as a drive shaft 210, a cam shaft 210, or an inner tube 210. The outer shaft 208 can also be referred to as an outer tube 208.

The blade 212a can be an elongate cutting member at a distal portion of the blade assembly 212. The blade 212a can include one or more sharpened edges configured to cut or resect tissue or other items. The blade assembly 212 can be located within the outer shaft 208 (and can be located within the inner shaft 210). The blade 212a can extend along (and optionally parallel with) the axis A1. The blade 212a can be translatable with respect to the inner shaft 210 and the outer shaft 208 to extend between (or into) the first jaw 206a and the second jaw 206b, such as along the blade slots 224a and 224b. In some examples, the blade 212a can extend axially through the inner shaft 210 offset from the axis A1. In some examples, the blade 212a the blade can extend axially through the flanges 220 and 222 such that the blade 212a is in a position laterally inward of the first set of flanges 220 and the second set of flanges 222.

The blade 212a can also be a translating member or electrosurgical component other than a blade. For example, the translating member 212a can be an electrode, such as a blunt electrode, a needle electrode, or a snare electrode.

The guide 218, the drive pin 216, and the pivot pin 214 can each be a rigid or semi-rigid pin, such as a cylindrical pin. The guide 218, the drive pin 216, and the pivot pin 214 can have other shapes in other examples, such as rectangular, square, oval, or the like. In some examples, the pivot pin 214 can have a size (such as a diameter) that is larger than the drive pin 216, as discussed below in further detail. Each pin can have a smooth surface to help reduce surface friction between the pins and components of the forceps 200, such as between the pivot pin 214 and the outer shaft 208 or the drive pin 216 and the flanges 220 and 222. Each of the guide 218, the drive pin 216, and the pivot pin 214 can be other components such as one or more projections, bosses, arms, or the like.

In operation, the inner shaft 210 can be translated using an actuator (such as the lever 116 of FIG. 1). The inner shaft 210 can translate with respect to the outer shaft 208 to move the drive pin 216. The drive pin 216 can engage the flanges 220 and 222 to move the flanges 220 and 222 between open and closed positions, which can cause the jaws 206a and 206b to move between open and closed positions.

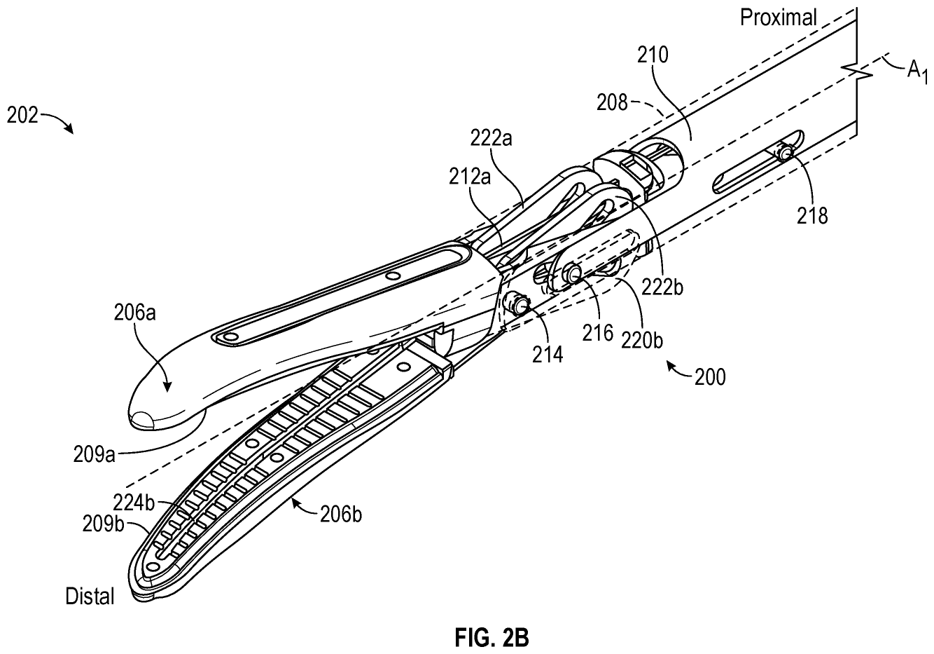

Though the forceps 200 are shown and discussed as being a cutting forceps or a coagulating forceps, the forceps 200 can be various other types of forceps or instruments used for grasping, cutting, measuring, coagulating, or the like. Further details and operation of the forceps 200 are discussed below with respect to FIGS. 3A and 3B.

FIG. 3A illustrates a side view of a portion of forceps 300 in a first electrode configuration. FIG. 3B illustrates a side view of a portion of the forceps 300 in a second electrode configuration. FIGS. 3A and 3B show orientation indicators Proximal and Distal and are discussed together below. The forceps 300 can be similar to the forceps 100 and 200 discussed above such that like numerals can represent like components; the forceps 300 can differ in that the forceps 300 can include segments (e.g., electrodes) configured to move relative to the jaws. Any of the forceps discussed above or below can be modified to include such features.

For example, the forceps 300 can include an end effector 302 that can include electrodes 309a and 309b connected to jaws 306a and 306b, respectively. The jaws 306a and 306b can include frames 307a and 307b, respectively. The frames can be rigid or semi-rigid bodies connected to flanges 320 and 322. The jaws 306a and 306b can also include housings 311a and 311b, respectively. The housings 311 can be over-molded to the frames 307 or otherwise connected thereto. The housings 311 can be rigid or semi-rigid bodies.

The electrodes 309 can be connected to the jaws 306 (e.g., to the frames 307 or housings 311) such that the electrodes move with the jaws 306 when the jaws are moved between the open and closed positions. The electrodes 109 can be connected (e.g., electrically) to a handpiece (e.g., 104) and an activation button or actuator (e.g. 122) such as via one or more wires. The electrodes 109 can be bipolar or monopolar. In some examples, the electrodes 309 can be moving segments, such as secondary frames or segments of the jaws 306 and can include or not include electrodes. The electrodes can be flexible, generally, and can be metal-impregnated polymer in some examples.

The electrodes 309 can also be connected, mechanically or electrically, to a second actuator (e.g., 120, 116, or an additional actuator). The first actuator, as discussed above, can be operable to activate the electrodes to coagulate or seal tissue or vessels. The second actuator can be operable to move the electrodes between an open position, as shown in FIG. 3A, and a closed position, as shown in FIG. 3B. The first actuator and second actuator of the electrodes can be operated independently or simultaneously.

FIGS. 3A and 3B also show that the flanges 320 and 322 can include tracks 323 and 324 that are configured to receive the cam pin 316 therein. During operation, the shaft (such as the inner shaft 110 of FIG. 1) can be connected to the cam pin 316 and can be operated to drive the cam pin in the tracks 323 and 324 to cause the flanges 320 and 322 to move, causing the jaws 306 to move between an open position, and a closed position, as discussed above with respect to FIGS. 2A-2C.

In operation of some examples, as shown in FIG. 3A, a vessel 50 can be positioned between the jaws 306a and 306b such that the electrodes 309a and 309b contact the vessel 60 and apply a pressure or force to maintain the vessel 50 within the jaws 306. Then, the electrodes 309a and 309b can be actuated or operated to move or change shape. For example, as shown in FIG. 3B, the electrodes 309a and 309b can curve with respect to the jaws 306a and 306b. Optionally, the electrodes 309 can curve until they engage each other. In some examples, the electrodes 309 can curve until just before they engage each other to help limit interaction between the electrodes 309 during operation of the electrodes 309 (e.g., use of electrosurgical energy for sealing or coagulating). In some examples, the end effector 302 can include insulators 332 on one or more ends (e.g., proximal and distal) of the electrodes. The insulators 332 can allow for the electrodes 309 to engage without impacting operation of the electrodes. In some examples, the insulators 332 can be omitted and the electrodes 309 can operate normally when in contact with each other.

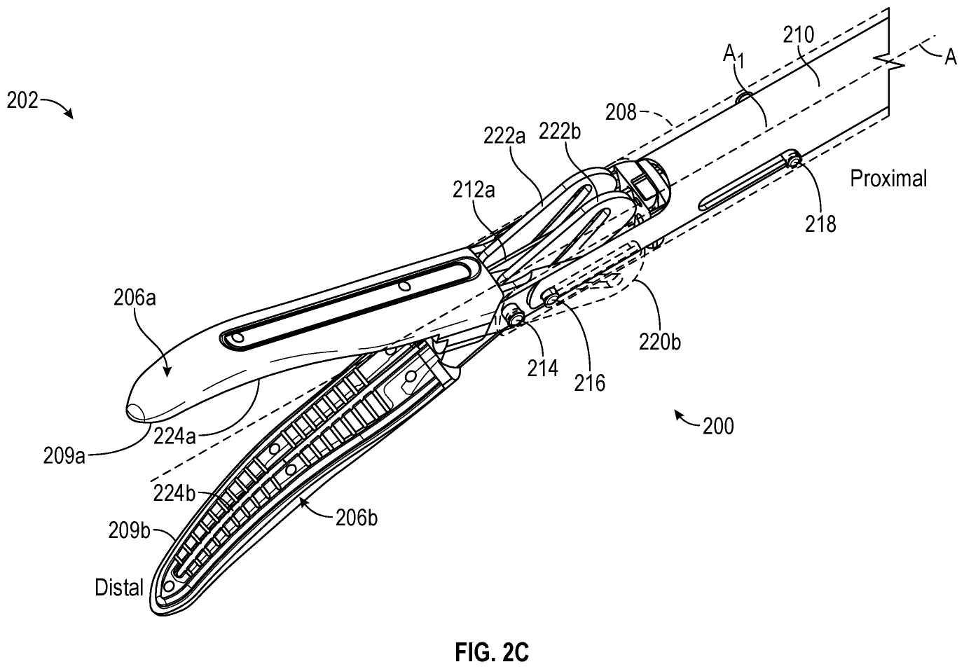

FIG. 4 illustrates a side view of a portion of forceps 400. FIG. 4 shows orientation indicators Proximal and Distal. The forceps 400 can be similar to the forceps 300 discussed above; the forceps 400 can differ in that the moving segments or electrodes can by segmented. Any of the forceps discussed above or below can be modified to include the features of the forceps 400.

For example, FIG. 4 shows electrode 409a that can include segments 434a, 434b, and 434c that can be connected by hinges 436a and 436b. That is, segment 434a is connected to segment 434b, at least in part, by hinge 436a, and segment 434b is connected to segment 434c, at least in part, by hinge 436b. The hinges 436 can allow the segments 434 of the electrode 409 to move or articulate relative to each other and to the jaw 406a. Similarly, the electrode 409b can includes segments 438a, 438b, and 438c connected by hinges 440a and 440b. Optionally, the segments 434a-434c can be connected to a face of an electrode where the electrode 409a is an electrode assembly. Optionally, the segments 434a-434c can be connected to an outer edge of an electrode where the electrode 409a is an electrode assembly.

The forceps 402 can also include actuators connected to the electrodes. For example, an actuator 442 can be connected to the electrode 409a. The actuator 442 can be a drive wire or similar element passing through at least a portion of the segments 434. The drive wire 442 can be operated (such as pushed or pulled) to cause the segments 434 to articulate. The drive wire 442 can extend through the shaft or shafts (e.g. 110 and 112) of the forceps to connect to an actuator for operation of the wire to articulate the electrode 409a. The electrode 409b can also be connected to such an actuator.

In operation of some examples, when the segments 434 articulate, the distal segment 434a can move such that a distal portion of the segment 434a can move laterally inward and the proximal segment 434c can move such that a proximal portion moves laterally inward. The shape formed by the articulated segments 434 can form around tissue, such as the vessel 50 to help apply homogenous pressure thereto. The segments 438 can form a similar but opposite (or mirror) shape to the segments 434 such that the vessel 50 can be contacted on multiple sides and effectively surrounded by the electrodes 409, which can help improve electrode operational efficiency (e.g., cutting, sealing, or coagulating).

Optionally, the electrode 409b can be connected to actuators 442a and 442b where the actuators 442a and 442b can be solenoids or other electromechanical linear actuators. The actuator 442b can include a base 444 and an armature 446. The base 444 (and a base of the actuator 442a) can be connected to the housing 411 (or frame) and can be electrically connected to a handpiece (e.g., 104) for receipt of electrical power therefrom. When actuated or powered, the base 444 can cause the armature 446 to extend therefrom (and relative to the housing 411) to engage the segment 438c, which can cause the segment 448c to extend or rotate about the hinge 440b. The segment 438a can be similarly operated by the actuator 442a. Movement of the segments 438 can help the segments 438 to apply a more uniform or homogenous pressure to the vessel 50.

Optionally, the actuators 442a and 442b can be operated independently, such as based on a shape or size of the tissue or vessel 50. For example, only the actuator 442a can be operated when the vessel 50 is relatively large. Optionally, the segments 438 can be biased to a neutral or open position (similar to FIG. 3A). Such a bias can be provided by the hinges 440, which can be sprung hinges, for example. Optionally, the segments or portions thereof can be made of a material with inherent biasing properties such as spring steel or Nitinol.

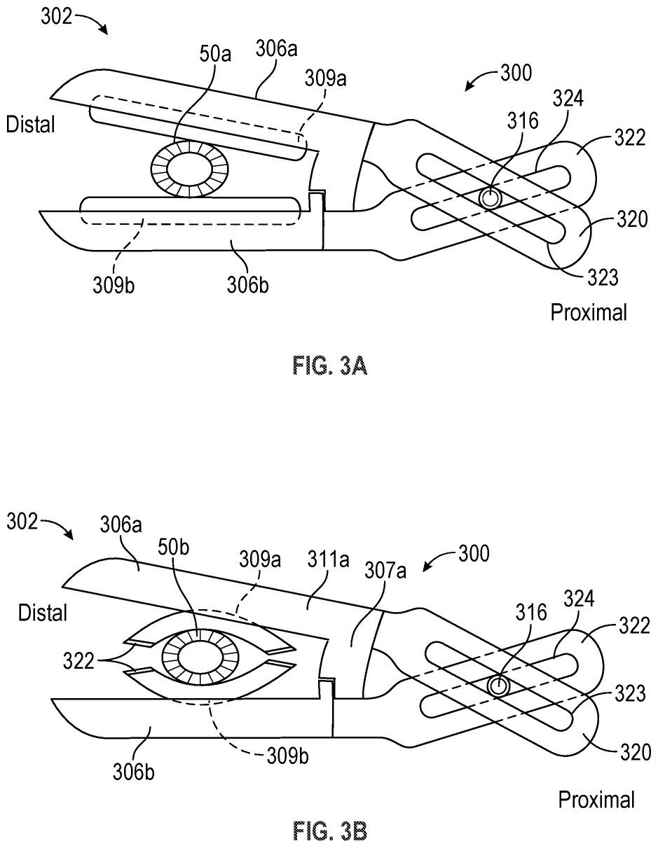

FIG. 5 illustrates a side view of a portion of forceps 500. FIG. 5 shows orientation indicators Proximal and Distal. The forceps 500 can be similar to the forceps 100-400 discussed above; the forceps 500 can differ in that one electrode can by segmented. Any of the forceps discussed above or below can be modified to include the features of the forceps 500.

That is, the electrode 509 can include a moving electrode 509a attached to a jaw 506 and a fixed electrode 509b attached to a jaw 506b. The jaws 560 themselves can be either moving or fixed. For example, the jaw 506a can be a moving or pivoting jaw and the jaw 506b can be fixed (or not pivoting). Optionally, the jaw 506b can be a moving or pivoting jaw and the jaw 506a can be fixed (or not pivoting). Optionally, both jaws 506a and 506b can be moving or pivoting jaws. That is either a moving or a fixed jaw can include an articulating or moving electrode.

The electrode 509a can include segments 534a and 534b that can be connected by one or more hinges 536. The forceps 500 can also include an actuator 542. The actuator 542 can be a drive wire or other actuator. Optionally, the actuator 542 can be an electromagnetic actuator (e.g., solenoid). The actuator 542 can be engaged or in contact with the segments 534 such that when the actuator 542 is operated, the actuator 542 can cause the segment 534a to move with respect to the segment 542b and the jaw 506a such that a distal end of the segment 534a moves laterally inward to close an opening between the electrodes 509. Such a forceps with only one moving electrode can provide the benefits of articulating electrodes while helping to limit cost.

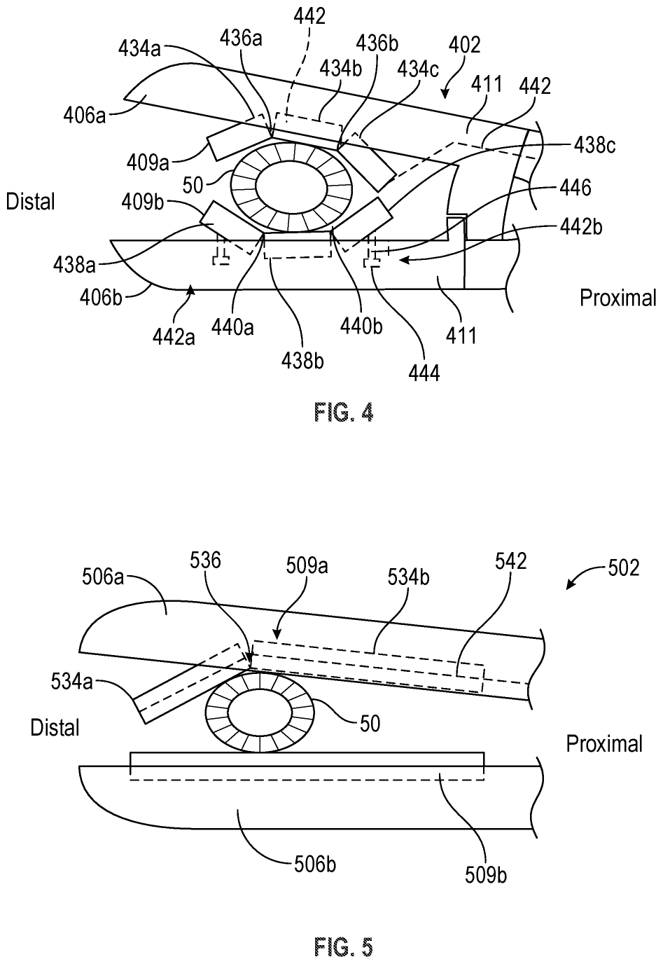

FIG. 6 illustrates a side view of a portion of forceps 600. FIG. 6 shows orientation indicators Proximal and Distal. The forceps 600 can be similar to the forceps 300-600 discussed above; the forceps 600 can differ in that a frame member or housing of the jaw can be flexible. Any of the forceps discussed above or below can be modified to include the features of the forceps 600.

The forceps 600 can include an end effector 604 that can include frame members 607a and 607b including flanges 620 and 622, respectively. The jaws 606a and 606b can also include housings 611a and 611b, respectively. The housings 611 can be connected to the frame members 607. The housing 611a can include an outer portion 646a and an inner portion 648a and the housing 611b can include an outer portion 646b and an inner portion 648b.

In some examples, the housings 611 can include an opening 650 or bore therein or therebetween. The opening 650a can be defined by the outer portion 646a and the inner portion 648a and the opening 650b can be defined by the outer portion 646b and the inner portion 648b. The opening 650a can be sized and shaped to receive the frame member 607a therein and the opening 650b can be sized and shaped to receive the frame member 607b therein, such as in an interference fit. In some examples, the housings 611 can be over-molded to the frame members 607. In some examples, the housings 611 can be fastened to the frame members, such as using one or more of screws, pins, adhesives, or the like.

The inner portions 648a and 648b can be configured to engage the vessel 50. Optionally, the inner portions 648 can include an electrode 609 embedded therein or thereon. The inner portions 648a and 648b can be made of a material configured to flex, such as one or more of a polymer, silicon, thing-gauge metal, or the like. In an example where the inner portions are made of thing gauge metal, the housings 611 can be hollow or incomplete.

The inner portions 648a and 648b can be configured to flex at hinge points 652a and 652b in response to a force F applied to one or more of the inner portions 648a and 648b. Such a force F can be caused by closing of the jaws 606 around the vessel 50 such as when the vessel 50 causes a reaction force to be applied to the inner portions 648. The reaction force F can cause the inner portion 648 to flex or hinge at the hinge points 652, allowing the housings 611 to change shape and confirm (or somewhat conform) to a shape of the vessel 50. Such a change in shape of the housings 611 can help to distribute pressure applied by the housings 611 to the vessel and can help to limit the vessel from ballooning proximally or distally. Optionally, the inner portions 648 can be formed to include multiple hinge of flex points.

Optionally, the jaws 606 can be or can include a single jaw member where the vessel 50 is manipulated by an arcing surface of a single member. For example, a member of the inner portion 648 or the frame or another component can roll, curl, or otherwise flex around the vessel 50 to secure or grasp the vessel.

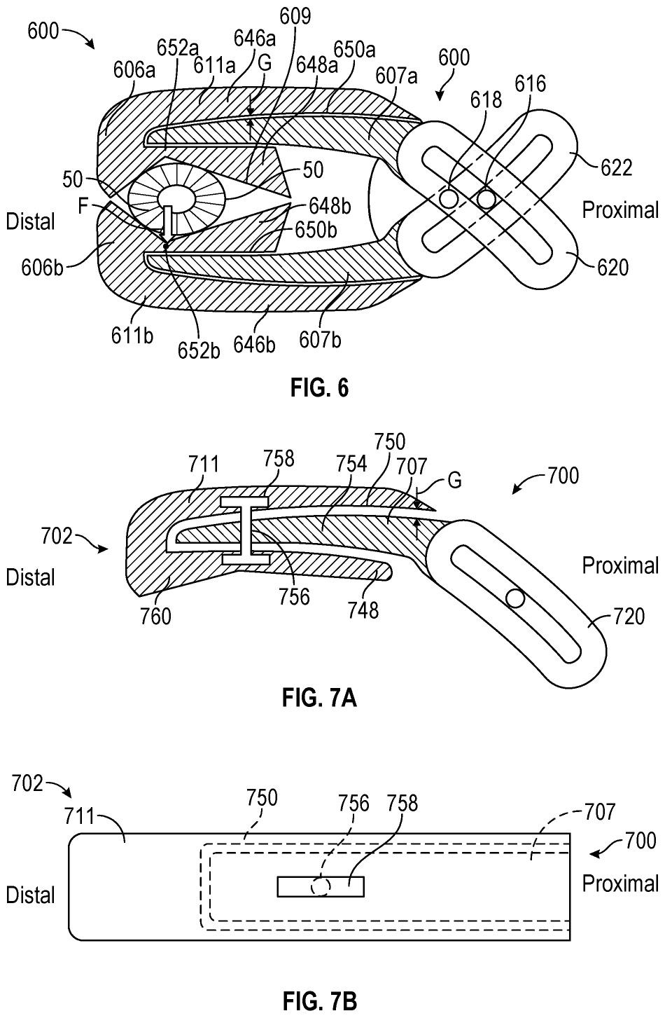

FIG. 7A illustrates a side view of a portion of forceps 700. FIG. 7B illustrates a top view of a portion of forceps. FIGS. 7A and 7B show orientation indicators Proximal and Distal. The forceps 700 can be similar to the forceps 100-600 discussed above; the forceps 700 can differ in that the housings can move relative to the frames. Any of the forceps discussed above or below can be modified to include the features of the forceps 700.

The forceps 700 and end effector 702 can include a frame member 707 that can include a flange 720 where the frame member 707 can be connected to a jaw 706. Though end effector 702 can include an additional framer member connected to the frame member 707 to form a pair of jaws, similar to any of those discussed above.

The frame member 707 can include a distal portion 754 that can be elongate and can extend from the flange 720. The distal portion 754 can include a bore 756 extending therethrough. The housing 711 can be a relatively rigid housing or can be flexible. The housing can define a proximal opening 750 extending into the housing 711 from proximally to distally. The proximal opening 750 can be sized and shaped to receive the distal portion 754 therein, which can be separated on one or both sides of the distal portion 754 by a gap G.

The frame member 707 can include a bore 756 extended through the distal portion 754. The bore 756 can be configured to receive a fastener 758 therethrough. The fastener 758 can be I-shaped and can be a two-piece (or more) fastener. In some examples, the fastener 758 can receive a screw or bolt therethrough to secure the fastener 758 together. The fastener 758 can secure the housing 711 to the frame member 707. Optionally, the bore 756 through which the fastener 758 extends can be relatively larger than the portion of the fastener 758 that extends through the bore such that the fastener 758 can move within the bore allowing relative movement of the housing 711 with respect to the frame 707.

In operation of some examples, the housing 711 can be configured to pivot or rotate about the fasteners 758 when a force is applied in a laterally outward direction on an inner portion 748 of the housing 711. Such a force can cause a distal end 760 of the housing 711 to move laterally inward with respect to the frame member 707. This movement or range of motion of the housing 711 with respect to the frame member 707 can be limited by the gap G between the frame member 707 and the housing 711. Laterally inward movement of the distal end 760 can allow a force or pressure applied by the jaw 706 to be applied more evenly and can help limit tissues or vessels from escaping outward from the jaw 706 distally during operations of the jaw 706.

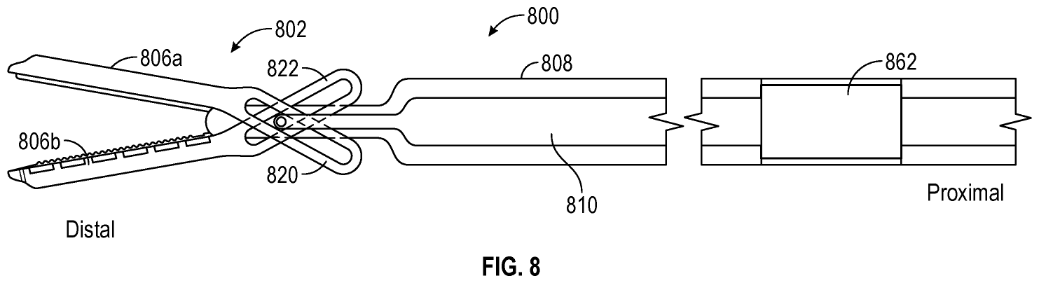

FIG. 8 illustrates a side view of a portion of forceps 800. FIG. 8 shows orientation indicators Proximal and Distal. The forceps 800 can be similar to the forceps 100-700 discussed above; the forceps 800 shows how the forceps 800 can include an actuator. Any of the forceps discussed above or below can be modified to include the features of the forceps 800.

The forceps 800 can include an end effector 802 including jaws 806a and 806b and flanges 820 and 822. The jaws 806 can be driven to open and close by translation of an internal shaft 810 with respect to an external shaft 808, similar to the forceps 100 and 200 discussed above. FIG. 8 shows an actuator 862 that can be connected to the inner shaft 810 or the outer shaft 808.

The actuator 862 can be an electromagnetic actuator connected to a handpiece or handle (e.g., 104). The actuator 862 can be located anywhere along a length of the shafts 808 and 810. The actuator 862 can be operated by one or more actuators of the handpiece and can be configured to, when operated, actuate one or more components of the end effector 802. For example, the actuator 862 can be connected to the inner shaft 810 and can cause the inner shaft 810 to translate to open and close the jaws 806. In an example where the outer shaft 808 translates, the actuator 862 can be configured to translate the shaft 808.

Optionally, the actuator 862 can be connected to actuators of the electrodes or segments, such as those described above. For example, the actuator 862 can be connected to one or more drive wires (e.g., 442 of FIG. 4) to cause the electrode (or segments thereof) to move or change shape with respect to the housing of the jaw.

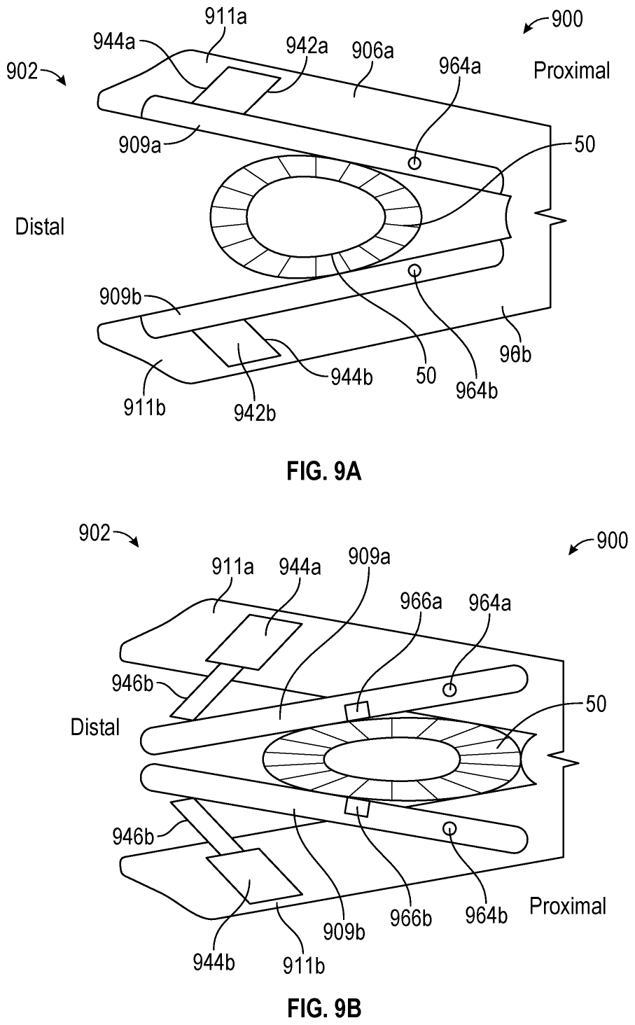

FIG. 9A illustrates a side view of a portion of forceps 900 in a first configuration. FIG. 9B illustrates a side view of a portion of forceps 900 in a second configuration. FIGS. 9A and 9B show orientation indicators Proximal and Distal. The forceps 900 can be similar to the forceps 100-800 discussed above; the forceps 900 can differ in that the jaws can be actuated in multiple ways. Any of the forceps discussed above or below can be modified to include the features of the forceps 900.

Jaws 906a and 906b can include segments 909a and 909b which can be electrodes, but can be other (non-electrode) segments or members of the jaws 906 that are configured to move. The segments 909a and 909b can be connected to housings 911a and 911b of the jaws at pivots 964a and 964b, respectively. The pivots 964 can be screws, rivets, pins or the like configured to connect the segments 909 to the housings 911 and configured to allow the segments 909 to pivot with respect to the housings 911.

FIGS. 9A and 9B also show actuators 942a and 942b that can be connected to the housings 911 and can be electrically connected to a handpiece (e.g., 104). The actuators 942 can be solenoids or other electromechanical linear actuators. The actuators 942 can include a base 944 and an armature 946. The bases 944a and 944b can be electrically connected to a handpiece (e.g., 104) for receipt of electrical power therefrom. When actuated or powered, the base 944a can cause the armature 946a to extend to engage a distal portion of the segment 909a, which can cause the segment 909a to extend or rotate about the pivot 964a. The segment 909b can be similarly operated by the actuator 442b.

The segment 909a can be pivoted about the pivot 964a such hat a distal portion of the segment 909a moves laterally inward and the segment 909b can be pivoted about the pivot 964a such hat a distal portion of the segment 909b moves laterally inward. Together, the segments 909 can move laterally inward to engage the vessel 50 to help limit the vessel from moving distally out of the jaws 906.

FIG. 9A also shows sensors 966a and 966b, which can be sensors of various types. For example, the sensors 966a and 966b can be force sensors, impedance sensors, resistance sensors, pressure sensors, optical sensors, or the like. The sensors 966a and 966b can be the same type of sensor, such as two impedance sensors. Optionally, the sensors 966a and 966b can be different types of sensors. For example, the sensor 966a can be an impedance sensor and the sensor 966b can be a force sensor.

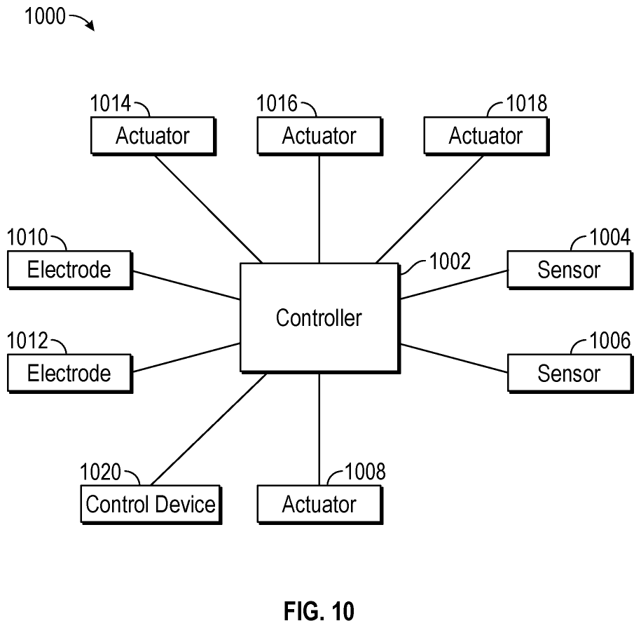

The actuators 942a and 942b can be configured to provide the same force F or can be configured such that the actuator 942a can provide or apply a force that is different from a force applied by the actuator 942b such that pressure applied to the vessel 50 can be varied or optimized such as based on feedback from the sensors 966a and 966b. Further details are discussed below with respect to FIG. 10.

FIG. 10 shows a schematic of a control system 1000 that can form all or a portion of a control circuit. The system 1000 can be configured to operate all or a portion of any of the forceps discussed above or below. The system 1000 can include a controller 1002, sensors 1004 and 1006, an actuator 1008, electrodes 1010 and 1012, and actuators 1014-1018.

The controller 1002 can be a programmable controller, such as a single or multi-board computer, a direct digital controller (DDC), a programmable logic controller (PLC), or the like. In other examples the controller 1002 can be any computing device, such as a handheld computer, for example, a smart phone, a tablet, a laptop, a desktop computer, or any other computing device including a processor, memory, and communication capabilities. The electrodes 1010 and 1012 can be connected to the controller 1002 and can be configured to deliver electrosurgical energy to tissue.

The sensors 1004 and 1006 can be connected to the controller 1002 and can be force sensors, impedance sensors, resistance sensors, pressure sensors, optical sensors, or the like. The sensors 1004 and 1006 can be the same type of sensor, such as two impedance sensors. Optionally, the sensors 1004 and 1006 can be different types of sensors. The sensors 1004 and 1006 can be connected to one or more components of any of the forceps discussed above. Though two sensors are shown, 1, 3, 4, 5, 6, 7, 8, 9, 10, 15, 20, or the like sensors can be included in the system 1000.

The actuators 1008 and 1014-1018 can be connected to the controller 1002 and can be electromagnetic actuators or other types of actuators. For example, any of the actuators of the system 1000 can be solenoids, servos, pistons, motors, Piezoelectric actuators, electroactive polymer actuators, or the like. The actuator 1008 can be one or more actuators and can be connected to a shaft of any of the forceps discussed above, such as the actuator 862 of FIG. 8. The actuators 1014-1018 can be one or more actuators. Though 3 actuators are shown, 1, 2, 4, 5, 6, 7, 8, 9, 10, 15, 20, or the like actuators can be connected to the controller 1002.

The actuators 1014-1018 can be connected to independent segments of a jaw. For example, the actuator 1014 can be connected to the segment 434a, the actuator 1016 can be connected to the segment 434b, and the actuator 1018 can be connected to the segment 434c. The actuators 1014-1018 can be operated independently or together in such a configuration. Optionally, the actuator 1014 can be connected to a segment (e.g., segment or electrode) of a first jaw and the actuator 1016 can be connected to a segment of a second jaw.

Any of the actuators of the control system 1000 can be part of an electromagnetic actuation system or circuit. For example, a first electromagnetic actuation system can include a first actuator (e.g., 1014) connected be in electrical communication with the controller 1002, and the control circuit or controller 1002 is configured to control the first electromagnetic actuation system using a stored first jaw pressure profile to provide localized jaw pressure.

In one example, the sensor 1004 can be a positions sensor configured to monitor a position of a jaw (e.g., 906a). The sensor 1004 can be configured to generate a signal representing a relative position between the first jaw and the second jaw (such as the frames or housings of the jaw). The stored first jaw actuation force profile can include a first jaw actuation force for a first relative position between the first jaw frame and the second jaw and a second jaw actuation force for a second relative position between the first rigid jaw frame and the second jaw.

FIG. 10 also shows a control device 1020 that can be connected to the controller and can be a handle, actuator, button, or the like, such as of a handpiece (e.g., 104). The control device 1020 can be in communication with the sensor 1004 (which can be a force sensor) and the force sensor 1004 can be configured to measure a force applied by a user to the handle. The first electromagnetic actuation system can be in electrical communication with the controller 1002 or control circuit, and the controller 1002 can be configured to control the first electromagnetic actuation system using the measured force. Optionally, the stored first jaw pressure profile can include a first jaw pressure for a first measured force and a second jaw pressure for a second measured force.

In one example, the controller 1002 can be in communication with a tissue sensor (e.g., 1004) and the tissue sensor can be configured to measure a characteristic of the object or the biological tissue. The first electromagnetic actuation system (e.g., 1014) can be in electrical communication with the controller 1002 and the controller can be configured to control the first electromagnetic actuation system using the measured characteristic. Optionally, the stored first jaw profile can include a first jaw pressure for a first measured characteristic and a second jaw pressure for a second measured characteristic, where the characteristic can be an impedance or phase angle. The actuation system can be operated by the controller 1002 and the control device 1020 based, at least in part, on the first characteristic, the second characteristic, the first jaw profile, or the second jaw profile.

Optionally, the characteristic can be resistance. For example, alternating current (AC) power can be used to power the electrodes and direct current (DC) power can be used to power the sensors 1004. In such an example, the first or second (or additional) characteristic can be resistance. This can be helpful, because, for example, DC may be more accurate or precise for controlling purposes while AC may be more useful for supplying power to the electrodes for coagulation functions. Optionally, DC power can be used for both the sensors and the electrodes.

Optionally, the sensors 1004 can be used to measure an angle of the jaws. For example, the sensors 966 can be used to measure an angle of the housings 911 or the grip plates 909 (or electrodes). For example, the controller 1002 can determine a position of the housings 911 based on feedback from the sensors (e.g., to determine sensor proximity). Optionally, the controller 1002 can determine a position of grip plates 909 (or electrodes) based and based on signals from the sensors 966 and whether the actuators 944 have been actuated. Optionally, the sensors 1004 can be used in place or in addition to the sensors 966.

In an example, an electromagnetic actuation system can include the actuator 1008 (e.g., actuator 862), which can be coupled with a movable handle (e.g., 116) and the first and second jaws (e.g., 806). The electromagnetic actuation system can be configured to assist a mechanical actuation system (e.g., shafts) in opening and closing of the first and second jaws. During such an operation, the rigid frames of the jaws can be moved relatively to one another to thereby open and close the jaws. Also, the actuator 1008 can be a solenoid coupled with an articulating drive shaft (e.g., inner shaft 110), wherein the articulating drive shaft can be connected to the first jaw and the second jaw.

In such an example, the sensor 1004 can be a position sensor that can be configured to measure a relative position of the movable handle (e.g., 116) and the electromagnetic actuation system can be configured to assist the mechanical actuation system in the opening and closing of the first and second jaws using the measured position of the movable handle.

In the same or another example, the sensor 1006 can be a position sensor configured to measure a relative position of one or more of the first and second jaws and the electromagnetic actuation system can be configured to assist the mechanical actuation system in the opening and closing of the first and second jaws using the measured position of the first jaw or the second jaw.

Optionally, the sensor 1004 can be a force sensor configured to measure a force applied by a user to the movable handle. The electromagnetic actuation system can be in electrical communication with the controller 1002, and the controller can be configured to control the electromagnetic actuation system using the measured force. In the same or another example, the sensor 1006 can be a force sensor coupled with a first electrode (e.g., 309) and the sensor can be configured to measure a force applied to the biological tissue (e.g., vessel 50) by the first electrode. And, the electromagnetic actuation system can be configured to use the measured force to assist the mechanical actuation system in opening and closing of the first and second jaws.

Optionally, the tissue sensor (e.g., 1004) can be configured to measure a characteristic of an object or a biological tissue and the controller 1002 can be configured to control the electromagnetic actuation system using the measured characteristic, such as an impedance or phase angle.

In one example, the sensor 1004 can be a force sensor configured to measure a force applied by a user to the movable handle and the sensor 1006 can be a jaw position sensor configured to generate a signal representing a relative position between the first jaw and the second jaw. The electromagnetic actuation system (e.g., the controller 1002 and the actuator 1008 or 1014) can be configured to select one or more of the first stored jaw pressure profile and the second stored jaw pressure profile. Optionally, the controller can be configured to select one of the first stored jaw pressure profile and the second stored jaw pressure profile using the measured force to control the electromagnetic actuation system. Optionally, the control circuit can be configured select one of the first stored jaw pressure profile and the second stored jaw pressure profile using the relative position between the first jaw and the second jaw to control the electromagnetic actuation system.

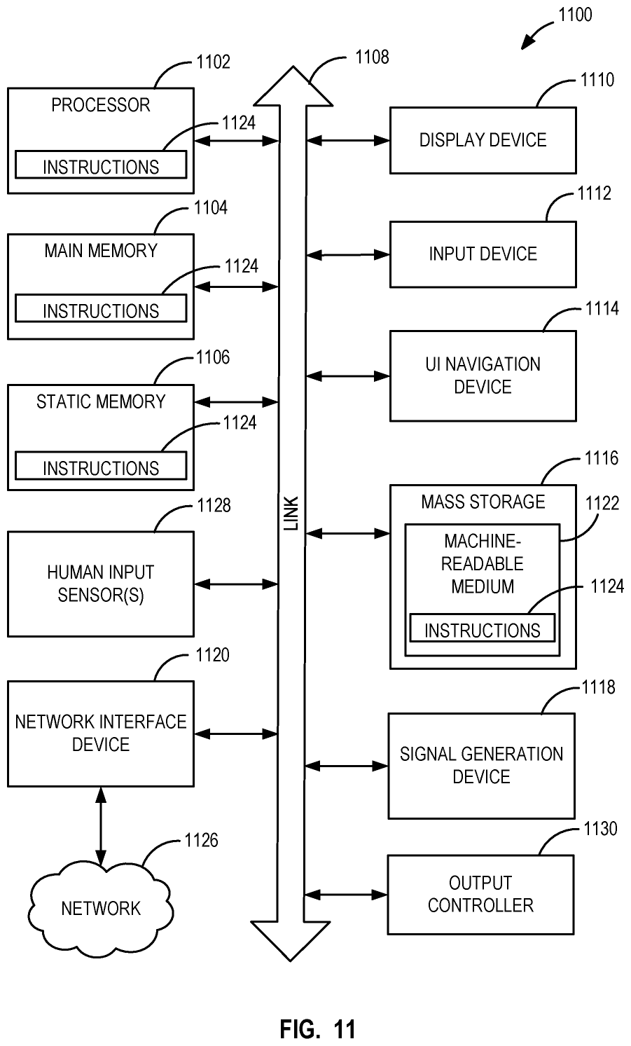

FIG. 11 illustrates a block diagram of an example machine 1100 upon which any one or more of the techniques (e.g., methodologies) discussed herein may perform. Examples, as described herein, may include, or may operate by, logic or a number of components, or mechanisms in the machine 1100. Circuitry (e.g., processing circuitry) is a collection of circuits implemented in tangible entities of the machine 1100 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1100 follow.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1100 may include a hardware processor 1102 (e.g., a central processing unit (CPU), a graphics processing unit (GPU), a hardware processor core, or any combination thereof), a main memory 1104, a static memory (e.g., memory or storage for firmware, microcode, a basic-input-output (BIOS), unified extensible firmware interface (UEFI), etc.) 1106, and mass storage 1108 (e.g., hard drive, tape drive, flash storage, or other block devices) some or all of which may communicate with each other via an interlink (e.g., bus) 1130. The machine 1100 may further include a display unit 1110, an alphanumeric input device 1112 (e.g., a keyboard), and a user interface (UI) navigation device 1114 (e.g., a mouse). In an example, the display unit 1110, input device 1112 and UI navigation device 1114 may be a touch screen display. The machine 1100 may additionally include a storage device (e.g., drive unit) 1108, a signal generation device 1118 (e.g., a speaker), a network interface device 1120, and one or more sensors 1116, such as a global positioning system (GPS) sensor, compass, accelerometer, or other sensor. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1102, the main memory 1104, the static memory 1106, or the mass storage 1108 may be, or include, a machine readable medium 1122 on which is stored one or more sets of data structures or instructions 1124 (e.g., software) embodying or utilized by any one or more of the techniques or functions described herein. The instructions 1124 may also reside, completely or at least partially, within any of registers of the processor 1102, the main memory 1104, the static memory 1106, or the mass storage 1108 during execution thereof by the machine 1100. In an example, one or any combination of the hardware processor 1102, the main memory 1104, the static memory 1106, or the mass storage 1108 may constitute the machine readable media 1122. While the machine readable medium 1122 is illustrated as a single medium, the term “machine readable medium” may include a single medium or multiple media (e.g., a centralized or distributed database, and/or associated caches and servers) configured to store the one or more instructions 1124.

The term “machine readable medium” may include any medium that is capable of storing, encoding, or carrying instructions for execution by the machine 1100 and that cause the machine 1100 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1124 may be further transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1100, and includes digital or analog communications signals or other intangible medium to facilitate communication of such software. A transmission medium is a machine readable medium.

### NOTES AND EXAMPLES

The following, non-limiting examples, detail certain aspects of the present subject matter to solve the challenges and provide the benefits discussed herein, among others.

Example 1 is a forceps comprising: a first jaw including a first jaw frame and a first movable jaw segment coupled with the first jaw frame; and a second jaw coupled with the first jaw, wherein the first movable jaw segment is in communication with a first electromagnetic actuation system, and wherein the first movable jaw segment is configured to be electromagnetically actuatable relative to the first jaw frame.

In Example 2, the subject matter of Example 1 optionally includes wherein the second jaw includes a second rigid jaw frame and a second movable jaw segment coupled with the second rigid jaw frame, and wherein the second movable jaw segment is in communication with a second electromagnetic actuation system, and wherein the second movable jaw segment is configured to be electromagnetically actuatable relative to the second rigid jaw frame.

In Example 3, the subject matter of Example 2 optionally includes wherein the first electromagnetic actuation system is configured to provide a first actuation force and the second electromagnetic actuation system is configured to provide a second actuation force different from the first actuation force.

In Example 4, the subject matter of any one or more of Examples 1-3 optionally include wherein the first jaw includes a first electrode configured to deliver electrosurgical energy to an object or biological tissue.

In Example 5, the subject matter of Example 4 optionally includes wherein the first movable jaw segment coupled with the first jaw frame is coupled with the first electrode.

In Example 6, the subject matter of Example 5 optionally includes wherein the first movable jaw segment coupled with the first electrode is coupled with a face of the first electrode.

In Example 7, the subject matter of any one or more of Examples 5-6 optionally include wherein the first movable jaw segment coupled with the first electrode is coupled with an outside edge of the first electrode.

In Example 8, the subject matter of any one or more of Examples 1-7 optionally include wherein the first movable jaw segment is movable between an open position and a closed position.

In Example 9, the subject matter of any one or more of Examples 1-8 optionally include wherein the first movable jaw segment is biased to an open position or a closed position.

In Example 10, the subject matter of any one or more of Examples 1-9 optionally include wherein the first movable jaw segment is positioned at a distal end of the first jaw.

In Example 11, the subject matter of any one or more of Examples 1-10 optionally include wherein the first electromagnetic actuation system is in electrical communication with a control circuit, and wherein the control circuit is configured to control the first electromagnetic actuation system using a stored first jaw force profile to provide a first localized jaw pressure.

In Example 12, the subject matter of Example 11 optionally includes a jaw position sensor configured to generate a signal representing a relative position between the first jaw frame and the second jaw, wherein the stored first jaw actuation force profile includes a first jaw actuation force for a first relative position between the first rigid jaw frame and the second jaw and a second jaw actuation force for a second relative position between the first rigid jaw frame and the second jaw.

In Example 13, the subject matter of any one or more of Examples 11-12 optionally include a handle in communication with a force sensor, the force sensor configured to measure a force applied by a user to the handle, wherein the first electromagnetic actuation system is in electrical communication with a control circuit, and wherein the control circuit is configured to control the first electromagnetic actuation system using the measured force.

In Example 14, the subject matter of Example 13 optionally includes wherein the stored first jaw pressure profile includes a first jaw pressure for a first measured force and a second jaw pressure for a second measured force.

In Example 15, the subject matter of any one or more of Examples 11-14 optionally include in communication with a tissue sensor, the tissue sensor configured to measure a characteristic of the object or the biological tissue, wherein the first electromagnetic actuation system is in electrical communication with a control circuit, and wherein the control circuit is configured to control the first electromagnetic actuation system using the measured characteristic.

In Example 16, the subject matter of Example 15 optionally includes wherein the stored first jaw profile includes a first jaw pressure for a first measured characteristic and a second jaw pressure for a second measured characteristic.

In Example 17, the subject matter of any one or more of Examples 15-16 optionally include wherein the characteristic is an impedance or phase angle.

Example 18 is a forceps comprising: a first jaw including a first rigid jaw frame; a second jaw coupled with the first jaw, the second jaw including a second rigid jaw frame; a movable handle; a mechanical actuation system coupled with the movable handle and the first and second jaws, the mechanical actuation system configured to control opening and closing the first and second jaws; and an electromagnetic actuation system coupled with the movable handle and the first and second jaws, the electromagnetic actuation system configured to assist the mechanical actuation system in opening and closing of the first and second jaws.

In Example 19, the subject matter of Example 18 optionally includes a fixed handle, wherein the electromagnetic actuation system includes an electromagnetic actuator positioned between the fixed handle and the movable handle.

In Example 20, the subject matter of any one or more of Examples 18-19 optionally include wherein the first jaw includes a first electrode configured to deliver electrosurgical energy to a biological tissue.

In Example 21, the subject matter of any one or more of Examples 18-20 optionally include wherein the first jaw includes a first jaw frame, and wherein the electromagnetic actuation system includes an electromagnetic actuator positioned between an electrode and the first jaw frame.

In Example 22, the subject matter of any one or more of Examples 18-21 optionally include wherein the electromagnetic actuation system includes a solenoid coupled with an articulating drive shaft, and wherein the articulating drive shaft is coupled with the first jaw and the second jaw.

In Example 23, the subject matter of any one or more of Examples 18-22 optionally include a position sensor configured to measure a relative position of the movable handle, wherein the electromagnetic actuation system is configured to assist the mechanical actuation system in the opening and closing of the first and second jaws using the measured position of the movable handle.

In Example 24, the subject matter of any one or more of Examples 18-23 optionally include a position sensor configured to measure a relative position of one of the first and second jaws, wherein the electromagnetic actuation system is configured to assist the mechanical actuation system in the opening and closing of the first and second jaws using the measured position of the one of the first and second jaws.

In Example 25, the subject matter of any one or more of Examples 18-24 optionally include a force sensor configured to measure a force applied by a user to the movable handle, wherein the electromagnetic actuation system is in electrical communication with a control circuit, and wherein the control circuit is configured to control the electromagnetic actuation system using the measured force.

In Example 26, the subject matter of any one or more of Examples 18-25 optionally include wherein the first rigid jaw frame includes a first electrode configured to deliver electrosurgical energy to a biological tissue, the forceps comprising: a force sensor coupled with the first electrode and configured to measure a force applied to the biological tissue by the first electrode, wherein the electromagnetic actuation system is configured to use the measured force to assist the mechanical actuation system in opening and closing of the first and second jaws.

In Example 27, the subject matter of any one or more of Examples 18-26 optionally include in communication with a tissue sensor, the tissue sensor configured to measure a characteristic of an object or a biological tissue, wherein the electromagnetic actuation system is in electrical communication with a control circuit, and wherein the control circuit is configured to control the electromagnetic actuation system using the measured characteristic.

In Example 28, the subject matter of Example 27 optionally includes wherein the characteristic is an impedance or phase angle.

In Example 29, the subject matter of any one or more of Examples 18-28 optionally include wherein the electromagnetic actuation system is in electrical communication with a control circuit, wherein the control circuit is configured to control the electromagnetic actuation system using a stored jaw pressure profile having a jaw pressure, wherein the stored jaw pressure profile includes a first stored jaw pressure profile having a first jaw pressure and a second stored jaw pressure profile having a second jaw pressure, and wherein the electromagnetic actuation system is configured to select one of the first stored jaw pressure profile and the second stored jaw pressure profile.

In Example 30, the subject matter of Example 29 optionally includes a force sensor configured to measure a force applied by a user to the movable handle, wherein the electromagnetic actuation system is in electrical communication with a control circuit, and wherein the control circuit is configured to select one of the first stored jaw pressure profile and the second stored jaw pressure profile using the measured force to control the electromagnetic actuation system.

In Example 31, the subject matter of any one or more of Examples 29-30 optionally include a jaw position sensor configured to generate a signal representing a relative position between the first jaw and the second jaw, wherein the electromagnetic actuation system is in electrical communication with a control circuit, and wherein the control circuit is configured select one of the first stored jaw pressure profile and the second stored jaw pressure profile using the relative position between the first jaw and the second jaw to control the electromagnetic actuation system.

In Example 32, the subject matter of any one or more of Examples 18-31 optionally include wherein the first jaw includes a first electrode configured to deliver electrosurgical energy to a biological tissue.

In Example 33, the subject matter of any one or more of Examples 18-32 optionally be in combination with any one or more of claim 1-17.

In Example 34, the apparatuses or method of any one or any combination of Examples 1-33 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A forceps comprising:
a first jaw including a first jaw frame and a first movable jaw segment coupled with the first jaw frame; and
a second jaw coupled with the first jaw,
wherein the first movable jaw segment is in communication with a first electromagnetic actuation system, and wherein the first movable jaw segment is configured to be electromagnetically actuatable relative to the first jaw frame.

2. The forceps of claim 1, wherein the second jaw includes a second rigid jaw frame and a second movable jaw segment coupled with the second rigid jaw frame, and
wherein the second movable jaw segment is in communication with a second electromagnetic actuation system, and wherein the second movable jaw segment is configured to be electromagnetically actuatable relative to the second rigid jaw frame.

3. The forceps of claim 2, wherein the first electromagnetic actuation system is configured to provide a first actuation force and the second electromagnetic actuation system is configured to provide a second actuation force different from the first actuation force.

4. The forceps of claim 1, wherein the first jaw includes a first electrode configured to deliver electrosurgical energy to an object or biological tissue.

5. The forceps of claim 4, wherein the first movable jaw segment coupled with the first jaw frame is coupled with the first electrode.

6. The forceps of claim 5, wherein the first movable jaw segment coupled with the first electrode is coupled with a face of the first electrode.

7. The forceps of claim 5, wherein the first movable jaw segment coupled with the first electrode is coupled with an outside edge of the first electrode.

8. The forceps of claim 1, wherein the first movable jaw segment is movable between an open position and a closed position.

9. The forceps of claim 1, wherein the first movable jaw segment is biased to an open position or a closed position.

10. The forceps of claim 1, wherein the first movable jaw segment is positioned at a distal end of the first jaw.

11. The forceps of claim 1, wherein the first electromagnetic actuation system is in electrical communication with a control circuit, and wherein the control circuit is configured to control the first electromagnetic actuation system using a stored first jaw force profile to provide a first localized jaw pressure.

12. The forceps of claim 11, comprising:
a jaw position sensor configured to generate a signal representing a relative position between the first jaw frame and the second jaw,
wherein the stored first jaw actuation force profile includes a first jaw actuation force for a first relative position between the first rigid jaw frame and the second jaw and a second jaw actuation force for a second relative position between the first rigid jaw frame and the second jaw.

13. The forceps of claim 11, comprising:
a handle in communication with a force sensor, the force sensor configured to measure a force applied by a user to the handle,
wherein the first electromagnetic actuation system is in electrical communication with a control circuit, and wherein the control circuit is configured to control the first electromagnetic actuation system using the measured force.

14. The forceps of claim 13, wherein the stored first jaw pressure profile includes a first jaw pressure for a first measured force and a second jaw pressure for a second measured force.

15. The forceps of claim 11, in communication with a tissue sensor, the tissue sensor configured to measure a characteristic of the object or the biological tissue,
wherein the first electromagnetic actuation system is in electrical communication with a control circuit, and wherein the control circuit is configured to control the first electromagnetic actuation system using the measured characteristic.

16. The forceps of claim 15, wherein the stored first jaw profile includes a first jaw pressure for a first measured characteristic and a second jaw pressure for a second measured characteristic.

17. The forceps of claim 15, wherein the characteristic is an impedance or phase angle.

18. A forceps comprising:
a first jaw including a first rigid jaw frame;
a second jaw coupled with the first jaw, the second jaw including a second rigid jaw frame;
a movable handle;
a mechanical actuation system coupled with the movable handle and the first and second jaws, the mechanical actuation system configured to control opening and closing the first and second jaws; and
an electromagnetic actuation system coupled with the movable handle and the first and second jaws, the electromagnetic actuation system configured to assist the mechanical actuation system in opening and closing of the first and second jaws.

19. The forceps of claim 18, comprising:
a fixed handle,
wherein the electromagnetic actuation system includes an electromagnetic actuator positioned between the fixed handle and the movable handle.

20. The forceps of claim 18, wherein the first jaw includes a first electrode configured to deliver electrosurgical energy to a biological tissue.
